(19) United States
(12) Patent Application Publication
(10) Pub. No.: US 2017/0304450 A1
(43) Pub. Date: Oct. 26, 2017

(54) PARENTERAL COMPOSITIONS AND USES THEREOF

(71) Applicant: CEVA SANTE ANIMALE, Libourne (FR)

(72) Inventors: Florence GUIMBERTEAU, Montussan (FR); Anne GENETEAU, Les Billaux (FR); Jerome GUYONNET, Ambares (FR)

(73) Assignee: CEVA SANTE ANIMALE, Libourne (FR)

(21) Appl. No.: 15/509,730

(22) PCT Filed: Sep. 9, 2015
(86) PCT No.: PCT/EP2015/070539

§ 371 Date: Mar. 8, 2017

(30) Foreign Application Priority Data
Sep. 9, 2014 (EP) 14306385.7

(51) Int. Cl. A61K 47/20; A61K 31/5415; A61K 31/192; A61K 31/165; A61K 9/08; A61K 31/7048; A61K 9/00

(52) U.S. Cl. CPC A61K 47/20; A61K 31/7048; A61K 31/5415; A61K 9/0019; A61K 31/165; A61K 9/08; A61K 31/192

(57) ABSTRACT

The present invention relates to a parenteral composition comprising an antibiotic and a non-steroidal anti-inflammatory agent in solution in a particular solvent. The composition is intended in particular to be injected in a non-human mammal, in particular a farm animal. It also relates to a method for treating disorders connected with a microbial infection in non-human mammals by injecting said composition.

The present invention relates to a parenteral composition comprising an antibiotic and a non-steroidal anti-inflammatory agent in solution in a particular solvent. The composition is intended in particular to be injected in a non-human mammal, in particular a farm animal. It also relates to a method for treating disorders connected with a microbial infection in non-human mammals by injecting said composition.

### TECHNOLOGICAL BACKGROUND

The parenteral route (such as the subcutaneous, intramuscular or intravenous route) is preferred for administering substances to non-human mammals, especially farm animals, for example cattle.

Intramuscular (IM) injections are used for medicinal products (antibiotics, anti-inflammatories etc.), certain antiparasitic agents, general tonics, certain vitamins and trace elements, hormones, anaesthetics, analgesics and vaccines. They are mainly performed in the muscular masses of the neck, where there is less risk of abscess and absorption is better owing to the constant movements of the neck. The substances are absorbed in the blood vessels of the connective tissue surrounding the muscle fibres. It is also possible to inject in the muscles of the flat part of the buttock or in the muscles of the back of the thigh. The maximum volume per injection site is generally from 25 to 30 mL for adult cattle, 10 to 20 mL for young cattle and 5 to 10 mL for calves. If the amount to be injected exceeds these volumes, the number of injection sites is increased, for example on either side of the neck, in order to ensure rapid and total absorption of the substance. Intramuscular injection must be done at a controlled rate so as not to tear the muscles.

Subcutaneous (SC) injections are used for administering sera (e.g. antitetanus serum), vaccines, certain internal antiparasitics, antibiotics and anti-inflammatories. Subcutaneous injections are performed at the front of the shoulder or on the flat of the neck. The product is deposited in the subcutaneous connective tissue, where it diffuses slowly. It is absorbed via the blood vessels. The amount of liquid injected subcutaneously is generally at most 50 mL in adult cattle, 20 mL in young cattle and 10 mL in calves. If a larger volume is to be injected, the injections will be distributed over several sites.

Intravenous (IV) injections are generally performed in the jugular vein of the neck, more rarely in the mammary vein. It is possible, for small amounts to be injected (less than 20 mL) and for large animals (over 300 kg), to inject in the medial caudal vein or artery, under the tail, at the level of the 3rd coccygeal vertebra. Intravenous injection must always be performed slowly, with products ideally warmed to a temperature of 20-30° C. If there are signs of intolerance (tremors, salivation, grinding of teeth, etc.), injection must be stopped, or continued even more slowly.

Injection of substances in non-human mammals, for example cattle, must be carried out correctly in order to avoid any injury to the animal. Generally it is done by the farmer and/or the veterinary surgeon. Moreover, parenteral injection may cause local inflammatory reactions at or around the injection site, which are reflected in the occurrence of pain and suffering, possibly causing partial or total physical incapacity of the animal.

Parenteral injections performed poorly or partially are reflected in poor absorption of the substances, leading to problems of adhering to the treatment or the presence of residues of medicinal products in the treated animal, with consequent delay or making its meat or its milk unsuitable for consumption.

One of the main difficulties in developing these parenteral compositions is the lack of knowledge about the general mechanisms of penetration of substances through the epidermis and the underlying layers of the skin. It is therefore particularly difficult to predict which compounds can be delivered by the parenteral route and which solvents will be suitable for this method of administration.

Moreover, the nature of the skin of non-human mammals explains why very few, if any, of the commercially available transdermal products for human use are effective.

Consequently, in the area of animal health and more particularly in the area of injectable medicines there is still a need for new stable liquid compositions and a method for stable parenteral liquid administration, which offer the user a safe and convenient means of administering a medicinal product while minimizing its residues and greatly reducing the animal's pain or stress.

In cattle, the general term “respiratory diseases” denotes a set of respiratory disorders that affect the lower respiratory tract, i.e. the lungs (pneumonia), or the upper respiratory tract (rhinitis, tracheitis, bronchitis). These diseases are generally caused by various pathogens and notably Gram-negative bacteria such as Pasteurella multocida, Mannheimia haemolytica, Histophilus somni, Mycoplasma bovis. These agents interact with one another or jointly with an inflammatory process or an allergic reaction, triggering the disease proper. The main clinical signs observed are fever, lethargy, loss of appetite and depression associated with more or less pronounced respiratory signs (shallow, rapid breathing, slight cough, nasal and ocular watery discharge and considerable salivation). These diseases may cause large economic losses in the farms affected, mainly owing to decreased body weight of the animals, increased mortality and the costs associated with the extra work (separation of the infected animals, treatment). Treatment must target the cause of the disease and the clinical signs. For this purpose, usually various specific treatments are administered sequentially or concomitantly, such as antibiotics, antiparasitics, non-steroidal anti-inflammatories, bronchodilators and mucolytics.

Mastitis is an inflammatory reaction of the mammary gland and is mainly of bacterial origin. It is of high prevalence among dairy cows and it represents one of the most important diseases in the dairy industry. The main pathogens responsible for mastitis are Gram-positive bacteria such as Staphylococcus aureus, Streptococcus uberis and Gram-negative bacteria such as Escherichia coli and chlamydia, as well as Mycoplasma bacteria. Once the bacteria have infected the mammary gland, it is necessary to eliminate the bacteria rapidly and effectively using antibiotic treatment by the systemic route that is appropriate to the clinical case and to reduce the inflammation using an anti-inflammatory.

Other non-human mammals, such as bovines, have pathologies of bacterial origin accompanied by inflammatory clinical signs that require sequential or concomitant administration of antibiotic and anti-inflammatory treatments, especially non-steroidal anti-inflammatories.

Today, about 50 antibiotics have received marketing authorization (MA). They are distributed in 11 classes, namely aminoglycosides, beta-lactams (penicillins/cephalosporins), amphenicols, tetracyclines, macrolides (and related substances), polypeptides, sulphonamides, quinolones, nitro-imidazoles, derivatives of nitrofurans and derivatives of the benzyl-pyrimidine ring. Antibiotics are generally bactericides or bacteriostatics directed against Gram-positive bacteria, for example Staphylococcus and Streptococcus, against Gram-negative bacteria, for example E. coli, Salmonella, Chlamydia, Citrobacter, Enterobacter, Serratias, Morganella, Proteus, Klebsiella, Shigella, Pasteurella, Mannheimia, Haemophilus, Moraxella, Pseudomonas, Brucella) or against Mycoplasma bacteria, for example mycoplasma.

The anti-inflammatories are intended for treating an inflammatory reaction and the diseases resulting therefrom. There are two main classes of anti-inflammatories: the glucocorticoids and the non-steroidal anti-inflammatories (NSAIDs). The NSAIDs are medicinal products with analgesic, antipyretic and/or anti-inflammatory properties.

A great many antibiotics in the form of liquid (transdermal) parenteral compositions are commercially available. We may mention as an example an amphenicol antibiotic, such as florfenicol marketed by the company Schering-Plough under the trade name Nuflor® described in patent U.S. Pat. No. 5,082,863, or forms suitable for intramuscular injection described in international application WO2004/014340 and application US2003/0068339.

Patent application WO2009/156369 describes liquid transdermal compositions comprising a non-steroidal anti-inflammatory drug (NSAID) and a pharmaceutically acceptable vehicle comprising a solvent and at least one skin penetration enhancer. This patent application illustrates compositions comprising meloxicam, ketoprofen and tolfenamic acid with a solvent system characterized by a skin penetration enhancer that is a mixture of L-menthol and Miglyol 840, and a solvent that is either diethylene glycol monoethyl ether (DEGMEE), or a mixture of isopropyl alcohol with 2-pyrrolidone or N-methylpyrrolidone. This liquid transdermal composition of an NSAID may optionally contain an antibiotic.

However, no result presented in this patent application can overcome the problems of pharmacokinetic interactions, absorption, distribution, biotransformation and/or excretion that are familiar to a person skilled in the art, which arise on administration of an NSAID and an antibiotic, as described in Benet L. et al. 1996: Pharmacological Basis of Therapeutics. pp. 3-28 and in Chaudary et al. 1999: Buffalo Bull. 18, 27-30 and Sharma et al. 2012: Vet. Archiv. 82, 555-565.

Therefore it is in order to overcome the problems of interactions encountered with the combination of non-steroidal anti-inflammatories (NSAIDs) and antibiotics, and to propose concentrated compositions so as to reduce their application volume, improve the well-being of non-human mammals by limiting the number of injection sites, while maintaining good stability during storage, and especially absence of formation of crystals, that the applicant has developed a composition that forms the subject matter of the invention. A particular aim was to propose a product for prophylactic and therapeutic treatment of bacterial infections, acting simultaneously on the cause of the disease and the clinical signs.

Another aim of the invention is to supply such compositions that can easily be administered at a single injection site whatever the animal species and its size.

Yet another aim of the invention is to supply, in a single liquid parenteral composition, a mixture of antibiotic and NSAID, without encountering problems of biopharmaceutical interactions between these substances.

In this connection, the applicant discovered that solubilization of an NSAID and an antibiotic, more particularly an amphenicol antibiotic and an oxicam, and even more preferably florfenicol and meloxicam, using a sufficient proportion of dimethylsulphoxide (DMSO) solvent, provided an effective solution to the problems encountered in the prior art. In fact, it was discovered, surprisingly, that formulation of an NSAID and an antibiotic, more particularly an amphenicol antibiotic and an oxicam, with at least 35% of DMSO, made it possible to solubilize higher concentrations of active ingredients without any risk of recrystallization. Addition of DMSO also offers the advantage that it is not necessary to provide special conditions for storage, such as storage at moderate temperatures above the freezing point. This is particularly surprising since DMSO is not generally selected as solvent for liquid formulations owing to its high freezing point, close to 18° C.

### SUMMARY OF THE INVENTION

The present invention proposes a veterinary composition comprising at least one antibiotic, at least one non-steroidal anti-inflammatory (NSAID) and a particular solvent, intended to be applied parenterally to non-human mammals, especially farm animals and/or domestic animals.

The parenteral composition according to the present invention has excellent stability. It is also practical to administer while minimizing the residues of active ingredients and/or reducing the animal's pain and/or stress.

The invention also relates to a method for treating disorders connected with microbial infection of non-human mammals, especially farm animals, by injecting said composition.

The invention also relates to the use of DMSO, at least one antibiotic, and at least one non-steroidal anti-inflammatory, for preparing an injectable veterinary composition, intended for treating disorders connected with microbial infections, and especially bacterial infections, in non-human mammals.

### DETAILED DESCRIPTION OF THE INVENTION

The present invention relates to a parenteral composition comprising an antibiotic, a non-steroidal anti-inflammatory (NSAID) and at least 35% of dimethylsulphoxide (DMSO).

The composition according to the invention is advantageously in liquid form.

In the present description, the percentages are expressed by weight (g) per volume (100 ml) of the composition, unless stated otherwise. Thus, 35% signifies that DMSO is present in an amount of 35 g to 100 ml of the composition.

The composition preferably comprises at least 40%, or 45%, or 50%, or 55% or 60% of DMSO.

Among the antibiotics, we may mention the antibiotics of the following classes: aminoglycosides, beta-lactams (penicillins/cephalosporins), amphenicols, tetracyclines, macrolides, polypeptides, sulphonamides, quinolones, nitro-imidazoles, derivatives of nitrofurans and derivatives of the benzyl-pyrimidine ring.

The beta-lactam class comprises the penicillins, among which we may mention group G, also called benzylpenicillin (Penicillin G), which notably comprises penicillin benzathine, benethacillin hydriodide or penethamate (penicillin ester); group A notably comprising the ampicillins, amoxycillin; and group M notably comprising cloxacillin, dicloxacillin, oxacillin and nafcillin. The cephalosporins mainly consist of cefalexin, cefalonium, cefapirinen, cefazolin, ceftiofur, cefoperazone, cefovecin or cefquinome. The aminoglycoside class notably comprises dihydrostreptomycin, neomycin, apramycin, gentamicin, kanamycin, spectinomycin and framycetin. The amphenicol class notably comprises chloramphenicol, thiamphenicol and florfenicol. The tetracycline class notably comprises tetracycline, oxytetracycline, chlortetracycline and doxycycline. The class of macrolides and related compounds notably comprises erythromycin, spiramycin, tylosin, josamycin, tilmicosin, tulathromycin, gamithromycin, tildipirosin, clindamycin, lincomycin, pirlimycin, tiamulin and valnemulin. The polypeptide class notably comprises colistin (Polymyxin E), colistimethate, polymyxin B and bacitracin. The quinolone class notably comprises oxolinic acid, flumequine, enrofloxacin, danofloxacin, ibafloxacin, marbofloxacin, difloxacin, orbifloxacin, rifampicin, rifaximine and pradofloxacin. The family of the sulphonamides notably comprises sulphamethizole, sulphathiazole, sulphadimidine (sulphadimerazine), sulphamethoxazole, sulphadiazine, sulphadimethoxine and sulphamethoxypyridazine. The class of the derivatives of the benzyl-pyrimidine ring notably comprises trimethoprim and baquiloprim. The nitrofuran class notably comprises nitrofurantoin, furazolidone and furaltadone. The nitro-imidazole class comprises for example metronidazole, dimetridazole and ronidazole.

The non-steroidal anti-inflammatory drugs (NSAIDs) notably comprise acetylsalicylic acid and acetylsalicylate lysine, salicylates such as notably methyl salicylate, arylacetic derivatives, or arylalkanoic derivatives, for example diclofenac, aceclofenac, sulindac or ketorolac (trometamol), 2-arylpropionic acids (profens), for example ibuprofen, ketoprofen, dexketoprofen, naproxen, oxaprozin and flurbiprofen, indole derivatives, for example indometacin (or indomethacin) or proglumetacin, oxicams, for example meloxicam, piroxicam and tenoxicam, nitrogen monoxide donor cyclooxygenase inhibitors (Cox inhibitors), for example naproxcinod, sulphonanilids, selective cyclooxygenase 2 inhibitors, for example celecoxib, etoricoxib, parecoxib, rofecoxib and valdecoxib, phenylbutazone, niflumic acid and N-arylanthranilic acids (phenamic acids).

Among the combinations of antibiotics and non-steroidal anti-inflammatory drugs according to the invention, we may notably mention the combination of an antibiotic of the amphenicol class (such as florfenicol) and a derivative of 2-arylpropionic acids (such as ketoprofen); the combination of an antibiotic of the amphenicol class (such as florfenicol) and an NSAID of the oxicam class (such as piroxicam or meloxicam); or the combination of an antibiotic of the macrolide class (such as tylosin) and a derivative of 2-arylpropionic acids (such as ketoprofen).

Preferably, the present invention relates to a parenteral composition comprising an antibiotic of the amphenicol class, an NSAID of the oxicam class and at least 35% of DMSO.

Preferably, the antibiotic is selected from the amphenicol antibiotics, in particular it is selected from chloramphenicol, thiamphenicol and florfenicol. The antibiotic in the present invention is more specifically florfenicol.

Among the non-steroidal anti-inflammatory drugs of the oxicam class, we may mention in particular meloxicam, piroxicam or tenoxicam. According to a specific embodiment, the non-steroidal anti-inflammatory drug of the oxicam class is meloxicam.

Even more preferably, the present invention relates to a parenteral composition comprising florfenicol, meloxicam and at least 35% of DMSO.

The composition according to the invention may optionally comprise other polar aprotic solvents. We may mention as examples of polar aprotic solvents selected from the alcohols, ketones such as acetone and butanone, from the N,N-disubstituted amines such as dimethylformamide (DMF), N-methyl-2-pyrrolidone (NMP), from the nitriles such as acetonitrile, from the esters such as ethyl acetate, propyl acetate, or amyl acetate, from the tertiary amines such as triethylamine, from the nitrogen-containing heterocycles, such as pyridine, or from the dioxolanes, such as glycerol formal.

According to a particular embodiment of the invention, the composition comprises at least one other solvent, and in particular at least one other polar aprotic solvent, and advantageously glycerol formal.

According to a particular embodiment, the composition according to the invention comprises two solvents, more specifically DMSO and glycerol formal.

Preferably, the antibiotic (more specifically florfenicol) is generally present in the composition according to the invention in a proportion between 20 and 60%, preferably between 30 and 50%, or between 35 and 45%, and more specifically 40%.

The non-steroidal anti-inflammatory drug (more specifically meloxicam) is generally present in the composition according to the invention in a proportion that may vary from 0.1 to 1%, preferably between 0.2 and 0.8%, and better still between 0.4 and 0.6%.

Generally, the compositions according to the invention are also called medicinal products or veterinary compositions or antibiotic pharmaceuticals.

The term “comprises” in the present invention may be interpreted more strictly; it may thus be replaced in particular embodiments by the terms “consists essentially of” or else “consists of”.

These veterinary compositions may further comprise a physiologically acceptable solvent and/or a physiologically acceptable excipient. “Physiologically acceptable” means a solvent or an excipient that is not harmful to the animal that is intended to receive the composition according to the invention. Thus, as described above, the compositions according to the invention may comprise a solvent, a pH regulator, a buffering agent, a suspending agent, a solubilizer, a stabilizer, a tonicity agent, an antioxidant and/or a preservative.

As other solvent, we may mention the oily solvents such as the medium-chain C₈-C₁₀ triglycerides, or a mixture of capric acid, caprylic acid and triglycerides, such as those that are marketed under the designation Mygliol®812, sesame oil, propylene glycol dicaprylocaprate, ethyl oleate.

Examples of suspending agents include methylcellulose, polysorbate 80, the sorbitan esters, hydroxyethylcellulose, xanthan gum, carboxymethylcellulose sodium and polyethoxylated sorbitan monolaurate. Examples of solubilizers include castor oil solidified with polyoxyethylene, polysorbate 80, nicotinamide, polyethoxylated sorbitan monolaurate, macrogol and the ethyl ester of castor oil fatty acid. Furthermore, the stabilizer includes sodium sulphite, sodium metasulphite and ether, ethylenediamine tetraacetic acid, whereas the preservative includes methyl p-hydroxybenzoate, ethyl p-hydroxybenzoate, sorbic acid, benzyl alcohol, phenol, cresol and chlorocresol. Among the antioxidants, we may mention as non-limiting examples: butylated hydroxytoluene, butylated hydroxyanisole, vitamin E, tert-butylhydroquinone and citric acid. An example of tonicity agent is mannitol.

During preparation of the injectable solutions or suspensions, it is desirable to ensure that they are isotonic with blood.

The proportions of the different elements included in the veterinary compositions according to the invention are determined by the standard practices familiar to a person skilled in the art who specializes in the formulation of pharmaceutical and veterinary products.

Preferably, the veterinary compositions according to the invention are administered by injection, in particular by intramuscular or subcutaneous injection. They are generally in the form of liquid solutions or suspensions. These antibiotic compositions can be stored for several months, namely 1 month, 2 months, 3 months and up to 6 months at room temperature or at a higher temperature of up to 40° C., and remain transparent and clear without degradation or formation of precipitate of the active principle or principles and more particularly of florfenicol and meloxicam.

Advantageously, the veterinary compositions may be administered in a single or in several injectable doses.

The injectable compositions are prepared by simple mixing of the components indicated above.

The present invention further relates to a kit for veterinary use, intended in particular for treating non-human mammals by injection, especially farm animals, against disorders connected with microbial infection. The kits according to the present invention comprise at least one compartment for optionally sterile packaging and comprising the composition according to the invention. The kit also comprises means for administering the compositions by injection, in particular by intramuscular or subcutaneous injection, such as notably at least one syringe and at least one needle, and optionally a leaflet of instructions on the method of use of the veterinary compositions according to the invention.

The composition according to the invention is intended in particular to be injected in a non-human mammal, in particular a farm animal or a companion animal.

It is particularly useful for treating disorders connected with microbial infections of non-human mammals, in particular of farm animals or of companion animals, by injecting said composition.

The farm animals or companion animals are in particular cattle, pigs, sheep, canines or felines, and more particularly cattle.

The farm animals are more specifically intended for consumption (for meat, offal or milk). They are in particular cattle, pigs or sheep. According to a particular embodiment, the farm animals are bullocks, cows (whether or not for milking) or pigs.

Domestic animals are more specifically companion animals, for example canines and felines.

The disorders connected with microbial, notably bacterial, infections may in particular be respiratory diseases that affect the lower respiratory tract, i.e. the lungs (pneumonia), or the upper respiratory tract (rhinitis, tracheitis, bronchitis), which may affect non-human mammals, and especially farm animals, such as cattle or sheep.

More specifically in cattle, the general term “respiratory diseases” denotes a set of respiratory disorders.

The treatment of microbial infections allows preventive or therapeutic treatment of respiratory diseases, in particular of cattle, or of mastitis, in particular in cattle, and more specifically in lactating cows.

Generally, in animal therapeutics, the veterinary surgeon or the farmer can determine the posology that he considers the most appropriate depending on whether it is prophylactic or therapeutic treatment, in relation to the animal's age and weight, and other particular factors of the subject to be treated. Administration is by means of systems for delivery and dosage that are known by a person skilled in the art.

Finally, the invention also relates to a method for treating disorders connected with microbial, in particular bacterial, infections of non-human mammals, in particular of farm animals or domestic animals, by injection of the composition according to the invention.

The invention is illustrated by the following examples, without limiting its scope.

### EXAMPLES

### Example 1

### Stabilities of Compositions Comprising a Non-Steroidal Anti-Inflammatory Drug and an Antibiotic

### Composition 1: Liquid Solution Comprising Florfenicol and Meloxicam

40 g of florfenicol and 0.5 g of meloxicam are dissolved in 45 g of dimethylsulphoxide (DMSO). Glycerol formal is added to give a final volume of 100 mL.

The solution thus obtained is stable physicochemically for at least 3 months at temperatures between 4 and 40° C.

### Composition 2: Liquid Solution Comprising Florfenicol and Ketoprofen

40 g of florfenicol and 3 g of ketoprofen are dissolved in 45 g of dimethylsulphoxide (DMSO). Glycerol formal is added to give a final volume of 100 mL. A clear colourless solution is thus obtained, which is stable physicochemically for at least 3 months at temperatures between 4 and 40° C.

### Composition 3: Liquid Solution Comprising Florfenicol and Piroxicam

40 g of florfenicol and 2.5 g of piroxicam are dissolved in 45 g of dimethylsulphoxide (DMSO). Glycerol formal is added to give a final volume of 100 mL. A clear, somewhat yellowish solution is thus obtained, which is stable physicochemically for at least 3 months at temperatures between 4 and 40° C.

### Composition 4: Liquid Solution Comprising Tylosin and Ketoprofen

8 g of tylosin tartrate and 2.4 g of ketoprofen are dissolved in 60 g of dimethylsulphoxide (DMSO). Glycerol formal is added to give a final volume of 100 mL. A clear, somewhat yellowish solution is thus obtained, which is stable physicochemically for at least 3 months at temperatures between 4 and 40° C.

### Composition 5: Liquid Solution Comprising Tylosin and Ketoprofen

8 g of tylosin tartrate and 2.4 g of ketoprofen are dissolved in 60 g of dimethylsulphoxide (DMSO). Ethyl acetate is added to give a final volume of 100 mL. A clear, somewhat yellowish solution is thus obtained, which is stable physicochemically for at least 3 months at temperatures between 4 and 40° C.

### Example 2

### Tests of Equivalence of Bioavailability

24 young male and female bovines aged 11 months and with an average weight of 220 kg are treated individually with 1 subcutaneous injection of 40 mg/kg of florfenicol (Nuflor 450®—Merck, 450 mg of florfenicol/mL). Blood samples are taken regularly during the 7 days following this injection. This is followed by a wash-out period of 20 days. The animals then receive a subcutaneous injection of 0.5 mg/kg meloxicam (Metacam®, Boehringer Ingelheim, 5 mg of meloxicam/mL). Blood samples are taken regularly during the 7 days following this injection. This is followed by a new wash-out period of 20 days. The animals then receive a subcutaneous injection of 1 mL/kg of Composition 1 (florfenicol and meloxicam). Blood samples are taken regularly during the 7 days following this injection. The blood samples obtained during this treatment campaign make it possible to evaluate the pharmacokinetic parameters (Cₘₐₓ and AUC₀₋ₜ) and a pharmacological parameter (useful life of the antibiotic) by conventional methods known by a person skilled in the art.

### Pharmacokinetic Parameters:

TABLE 1
Determination of Cₘₐₓ and plasma AUC of florfenicol

| | Florfenicol | Composition 1 |
|---|---|---|
| Cₘₐₓ (μg/L) | 3500 ± 1035 | 4647 ± 1597 |
| AUC₀₋ₜ (h * μg/L) | 196289 ± 44987 | 228139 ± 48084 |

TABLE 2
Determination of Cₘₐₓ and plasma AUC of meloxicam

| | Meloxicam | Composition 1 |
|---|---|---|
| Cₘₐₓ (μg/L) | 2239 ± 357 | 2011 ± 338 |
| AUC₀₋ₜ (h * μg/L) | 40569 ± 7122 | 34605 ± 5860 |

### Pharmacological Parameter:

The useful life of an antibiotic is the time (in hours) during which the plasma concentration of a given antibiotic is above its MIC₉₀ for the 3 representative microbes Mannheimia haemolytica, Histophilus somni, Pasteurella multocida.

The reference MIC₉₀ values for florfenicol on these 3 reference strains are: 1 μg/mL for Mannheimia haemolytica, 0.2 μg/mL for Histophilus somni and 0.5 μg/mL for Pasteurella multocida.

TABLE 3
Calculated antibiotic life (expressed in hours)

| | Composition 1 | Florfenicol |
|---|---|---|
| Mannheimia haemolytica | 72.08 ± 18.23 | 66.44 ± 20.65 |
| Histophilus somni | 160.48 ± 14.72 | 167.78 ± 0.13 |
| Pasteurella multocida | 120.40 ± 24.78 | 132.03 ± 27.22 |

The principal pharmacokinetic and pharmacological parameters of florfenicol are comparable, and thus demonstrate comparable exposure and efficacy of the antibiotic between the formula containing florfenicol or meloxicam alone and the compositions according to the invention.

1-10. (canceled)

11. A parenteral veterinary composition comprising a non-steroidal anti-inflammatory drug, an antibiotic and at least 35% of dimethylsulphoxide.

12. The composition according to claim 11, wherein the non-steroidal anti-inflammatory drug is of the oxicam class, preferably selected from meloxicam, piroxicam or tenoxicam, and in particular meloxicam.

13. The composition according to claim 11, wherein the antibiotic is selected from the amphenicol antibiotics, in particular is selected from chloramphenicol, thiamphenicol and florfenicol and in particular is florfenicol.

14. The composition according to claim 11, said composition comprising at least one other solvent, and in particular at least one other polar aprotic solvent, and advantageously glycerol formal.

15. The composition according to claim 11, said composition being suitable for injection into a non-human mammal, in particular a domestic animal or a farm animal.

16. The composition according to claim 11, wherein said composition is suitable for injection into a domestic animal or a farm animal, and wherein said animals are cattle, pigs, sheep, canines or felines, and in particular cattle.

17. The composition according to claim 11, wherein said composition is suitable for intramuscular or subcutaneous injection into a non-human mammal.

18. A method for the treatment of disorders connected with microbial infections of non-human mammals, by injecting a parenteral veterinary composition comprising a non-steroidal anti-inflammatory drug, an antibiotic and at least 35% of dimethylsulphoxide.

19. The method according to claim 18, wherein the disorders connected with microbial infections are respiratory diseases or disorders that affect the lower or upper respiratory tract.

20. A kit for veterinary use comprising a parenteral veterinary composition comprising a non-steroidal anti-inflammatory drug, an antibiotic and at least 35% of dimethylsulphoxide and at least one compartment for optionally sterile packaging.
